(19) United States
(12) Patent Application Publication
(10) Pub. No.: US 2008/0118615 A1
(43) Pub. Date: May 22, 2008

(54) Method for Analysing and Treating Human Milk and System Therefore

(75) Inventors: Peter Edwin Hartmann, Gooseberry Hill (AU); Ching Tat Lai, Canning Vale (AU); Jillian Lois Sherriff, Crawley (AU); Karen Norrie Simmer, Claremont (AU); Michelle Anne Lewis, Atwell (AU); Leon Robert Mitoulas, Cham (CH); Bronwyn Isabelle Davis, Queensland (AU)

Correspondence Address: Michael H Baniak;Mcdonnell Boehnen Hulbert&Berghoff LLP, 300 South Wacker Drive, Suite 3100, Chicago, IL, 60606, US

(73) Assignee: Medela Holding AG, Baar (CH)

(21) Appl. No.: 11/662,349

(22) PCT Filed: Sep. 2, 2005
(86) PCT No.: PCT/CH05/00519

§ 371 Date: Nov. 1, 2007

(30) Foreign Application Priority Data
Sep. 10, 2004 (EP) 04405566.3
Sep. 17, 2004 (EP) 04405591.1

(51) Int. Cl. G01N 33/04; G01N 33/14; G06F 19/00

(52) U.S. Cl. 426/231; 702/19

(57) ABSTRACT

The present invention relates to a method for analysing and treating human milk to be fed to an infant comprising the steps of collecting own mother's milk, taking a sample of the collected own mother's milk, conducting nutritional analysis on said sample, using the collected own mother's milk as nutrition for the infant and using the collected own mother's milk in the form of at least one of the group of: unchanged own mother's milk, fortified own mother's milk, unchanged components of own mother's milk and fortified components of own mother's milk, wherein said form is chosen depending on at least some of said results of the analysis and said infant's condition, the infants condition being chosen from at least one of the following parameters: infant's age, infant's weight, infant's health, infant's shortcomings, infant's deficiencies, time of day when the milk is fed to the infant.

### TECHNICAL FIELD

The present invention relates to a method for analysing and treating human milk to be fed to an infant. It furthermore relates to a system for processing results of nutritional analysis of a sample of collected human milk.

### BACKGROUND OF THE INVENTION

Human milk is commonly recognized as the optimum feeding for infants due to its nutritional composition and immunological advantages. Milk from the infant's own mother is considered a desirable feeding for infants of all ages, but also for preterm, low-birth-weight infants in early newborn intensive care units.

If the infant can not be fed with his own mother's milk, donor milk is considered to be second best. However, it was found that donor milk does not always supply the appropriate mixture of nutrients and immunological components, especially for preterm infants.

It was also found, that preterm human milk is apparently lacking in several constituents such as calcium, phosphorus and protein. Thus, it has been recommended that when preterm infants are fed preterm human milk, the human milk be fortified to better meet the nutritional needs of the preterm infant. Liquid and powder forms of preterm milk fortifiers have been marketed domestically in response to this recognized need. They not only comprise protein, fat and carbohydrates, but also substantial amounts of minerals as well as vitamins. The fortifiers differ with respect to their form, source of ingredients and energy and nutrient composition. Generally one can say that powder products are advantageous to minimize the dilution of mother's milk, while if mom's milk supply is limited, a liquid fortifier may be used to stretch her supply of human milk.

Under certain conditions also term infants during some stage of their development need additives to the milk or supplemental feeding to get the optimum nutritional conditions for ideal growth and best resistance to illnesses.

Herewith explicit reference shall be made to a very comprehensive, detailed and general description of the problems and the present status of development in the field of human milk as given in the background portion of U.S. Pat. No. 6,294,206.

Furthermore, US 2002/0182243 discloses methods for isolating human milk comprising the steps of collecting a sample of human milk from a donor in a collection device, storing the sample of milk obtained from the donor and processing the milk sample by conducting a nutritional analysis on the milk sample, fortifying the sample with heat-resistant nutrients, pasteurising the sample, fortifying the pasteurised sample with heat-sensitive nutrients and testing the sample to ensure successful pasteurisation. The obtained product is stored in a milk bank for later use as nutrition for an infant. US 2002/0182243 also suggests to test and process mother's own milk at the hospital, designed to screen for the presence of the most common pathogens, drugs and contaminants. The mother's milk, intended for her own baby, is housed in a milk laboratory under optimum storage conditions.

The prior art therefore either suggests to use donor milk, which is well analysed and fortified, or to use mother's own milk. If own mother's milk is used, only the pre-mature milk is fortified.

### SUMMARY OF THE INVENTION

It is an aim of the present invention to improve the nutrition of an infant.

This aim is achieved by a method for analysing and treating human milk to be fed to an infant comprising the steps of
collecting own mother's milk, taking a sample of the collected own mother's milk,
conducting nutritional analysis on said sample, using the collected own mother's milk as nutrition for the infant and
using the collected own mother's milk in the form of at least one of the group of: unchanged own mother's milk, fortified own mother's milk, enriched own mother's milk, deriched own mother's milk; wherein said form is chosen depending on at least some of said results of the analysis and said infant's condition, the infants condition being chosen from at least one of the following parameters: infant's age, infant's weight, infant's health, infant's shortcomings, infant's deficiencies, time of day when the milk is fed to the infant.

“Deriched” is used as an antonym to “enriched”. It is depleted milk or milk of diminished constitution.

A preferred method which is carried out in an automatic manner or semi-automatic manner is carried out using the following steps:
1. mother's milk is collected after input, weighted and at least one sample taken for analysis, optionally including spectroscopic and/or microbial analysis;
2. the remaining mother's milk is stored and frozen, which step may take place in the collection container;
3. the samples taken are analysed preferably by means of sensors including spectroscopic sensors, microbial assays and the like (for example mastitis checks etc), this step takes place while the remaining mother's milk is kept in stored and frozen state (see step 2);
4. the data of the analysis are input into a data processing unit and therein compared with reference data available from a database (for example in the form of a lookup table), including general data (general information later about ideal composition of milk as a function of parameters of the childlike weight, age, health status etc) as well as individual data about the baby to be fed (actual weight, age, health status etc, particular events), and determination of a mother's milk modification scheme (determination of necessary deriching and/or fortification in case of necessary modification to obtain ideal milk composition);
5. if clearance is given based on the evaluation of the data of the analysis (in particular if the microbial analysis leads to the result that the milk can be used for feeding), the remaining mother's milk is thawed by heating and/or crushing;
6. based on the modification scheme, the remaining mother's milk is then
   a. deriched by centrifugation and/or filtration and the like
   b. mixed and/or, homogenized by sonication, pressure, jet injection and the like
and/or pasteurised
and/or fortified;
and a sample taken for an additional, preferentially microbial analysis;
7. the modified mother's milk is then frozen and stored, if need be labelled, and subsequently either kept in frozen state or thawed by means of heating and/or crushing and output for use for feeding;
   all of the above steps being carried out automatically, and all of the steps preferably being tempered to be at a temperature of not more than 10° C. (apart from certain heating steps for thawing).

The invention further relates to a system for processing results of nutritional analysis of
a sample of collected human milk comprising
a data processing device,
stored data related to recommended compositions of human milk which consider nutritional needs of infants in different conditions, the infants conditions being chosen from at least one of the following parameters: infant's age, infant's weight, infant's health, infant's shortcomings, infant's deficiencies, time of day when the milk is fed to the infant,
input means for entering data related to an infant to be fed with this collected human milk and
means for evaluating a recommended composition for said infant out of the group of said stored data related to recommended compositions,
means for comparing said evaluated recommended composition with said results of nutritional analysis,
means for evaluating changes to be made to the collected human milk in order to obtain a recommended composition and
output means for showing said changes to be made.

The analysis performed in the state of the art were performed on donor human milk and own mother's milk out of concerns about possible drugs, bacterial, viral and other contaminations. Further testing of own mother's milk was performed for research reasons in order to improve the knowledge about optimised nutrition of infants in general.

According to the process of this invention however, a sample of collected own mother's milk is analysed and the remaining collected own mother's milk is treated in such a way as to provide an optimised nutrition to the infant. This means, that each time before the infant is feed or at least on a periodically basis, the own mother's milk is tested and treated in order to meet the changing requirements of the infant. These requirements change with the development of the infant. They can however also be different depending on the time of day the infant is fed. Furthermore, they depend on the health state of the infant, for example if the infant has a cold, gets a new tooth or if he just grows quite quickly at the moment. The inventive process also allows to consider that the composition of the mother's milk can vary. It varies for example depending on the time of day, on the mother's health and on her diet.

The inventive process enables to combine the advantages of own mother's milk, such as the immunology advantages, with the advantages of modem design of human milk to improve nutrition of an infant, using for example fortifiers. Another advantage of the inventive process is, that changes in the mother's milk caused by the diet of the mother can be detected and the milk can be treated accordingly, giving the child the components needed and not being only dependent on the mother's diet.

In a first embodiment of the present invention, at least one data table is provided which lists recommended compositions of human milk considering the nutritional needs of infants in different stages of developments for healthy infants and for infants with diseases or shortcomings and which lists possible compositions of human milk and changes to be made thereof to achieve these recommended compositions.

According to a preferred embodiment of the invention, the results of the analysis are automatically processed in a data processing device and a recommendation how to use the own mother's milk for nutrition of the infant is given as output data.

According to another preferred embodiment of the invention, the analysis of the own mother's milk is performed automatically. In another preferred embodiment, the automatic analysis and processing of the results is done within the same apparatus.

According to a further preferred embodiment, there is provided a fully automated and integrated device for handling mother's milk. Such a device comprises the following elements:
a milk container with a scale or other means for weighting milk contained in said milk container,
analysing means for taking samples from said milk container and for analysing the samples;
a data processing unit for interpreting the data obtained from the analysing means for the determination of a modification/storage/further use scheme for the remaining mother's milk, and for controlling further handling and fortification of the remaining mother's milk;
a first unit for storage and freezing during analysis of the remaining mother's milk, wherein this unit may be realised in the form of the above milk container with corresponding freezing means like cooling circuit, cooling coils and the like;
a first unit for thawing the remaining mother's milk, preferably including heating and/or crushing means, wherein this unit may also be realised as accessory to the above milk container, i.e. the above milk container may be provided with corresponding heating means, mechanical crushing elements and the like;
a unit for deriching the remaining mother's milk, preferably including centrifugation and/or filtering means, wherein for example centrifugation may also be carried out using the same container as given above milk container;
a unit for homogenisation of the deriched mother's milk, preferably including sonication, pressure and/or jet injection means, wherein this element may be realised for above milk container;
a unit for pasteurization;
a unit for fortification including input means for at least one fortifier and including means for taking at least one sample for additional microbial analysis;
a second unit for storage and freezing, wherein this unit may be realised in the form of the above milk container with corresponding freezing means like cooling circuit, cooling coils and the like;
optionally a unit for labelling of samples;
a second unit for thawing of the modified mother's milk, preferably including heating and/or crushing means, wherein for example centrifugation may also be carried out using the same container as given above milk container;
an output device for the modified mother's milk.

Preferably, the analysing means include microbial analysis and/or a spectrometer, and/or sensors like bio-sensors for the determination of specific metal contents, protein contents, and the like.

Preferably tempering means are provided in such a fully integrated and automated device such that in the milk container and in or during each unit a temperature of not more than 10° C. is safeguarded. This is to keep controlled conditions while treating and storing the sample.

The handling of the milk in the device may be structured in different ways. One possibility is that there is provided one container, which is subject to the individual steps or units as given above, so such a container will be provided with different interfaces like possibilities for heating, cooling, mechanical interfaces like crushing means, means for centrifugation of the container, tubing for taking out milk, tubing for introducing solids or liquids (fortifier and the like) into the container etc. In this case, for each use one standardised container is introduced into the device, preferably using special plug in connections, and the whole handling basically takes place around this single container. The advantage of this setup is that only one container has to be provided which can be cleaned easily, and that there is only as little as possible tubing which might have to be cleaned after treatment. A disadvantage of such a system might be that it is not possible to treat several samples subsequently within a short amount of time, since only one batch can be treated in one container.

In the alternative and according to another preferred embodiment of the integrated and automated device, there is provided several containers in which at least one or several of the above units are realised, and wherein transport of the milk between these containers or units is provided by means of tubing. In this case, preferentially means are provided to wash said tubing between individual transfer steps of mother's milk.

Such a fully automated device may for example be used in particular when feeding preterm infants, where very often the mother's milk has to be modified for proper feeding of the preterm child. The device may either be used for one single mother only, and in this case it might be sufficient to provide input once, updating it as a function of the development of the child and the mother, for example by using the input keyboard. Such an integrated device may however also be very useful for a hospital use, and in this case usually a large number of mothers is going to use one single device. In this case, either a database for individual mothers can be provided in the data processing unit, or each mother may be provided with a memory card carrying the individual information about the mother and the child. Therefore, the input possibilities for such an automated and integrated device may be given by a keyboard and/or by an interface such as a memory slot. Furthermore, preferentially data output means are provided, which are for example given by a printer and/or a display.

According to a preferred embodiment of the invention, the own mother's milk is at least analysed in view of its content of calcium and phosphorus. An analysis for proteins can be performed as well. Other components which can be analysed are fat, protein and lactose. Milkoscan can be used to determine these three macro nutrients in human milk. sIgA, lactoferrin, lysozyme, alpha-lactalbumin, serum albumin, beta casein, kappa casein, c3 and c4 complement can be measured at once with a method called ‘Nephelometric Immunoassays’. This method is described in Montagne et al. 2000, “Measurement of nine human milk proteins by nephelometric immunoassays” in Application to the determination of mature milk protein profile, clinical biochemistry 33(30) 181-186. Zinc, sodium, chloride, fatty acid composition, oligosaccharide can be measured as well.

Further embodiments of the present invention are outlined in the dependent claims.

### DETAILED DESCRIPTION OF THE PREFERRED EMBODIMENTS

According to the invention, own mother's milk is collected before feeding an infant using a breast pump known in the state of the art. The collected milk is stored in a storage according to the state of the art. A sample is taken from the collected milk and nutritional analysis on said sample is conducted. At least the content of calcium and phosphorus are detected in this sample. Preferably, the analysis is performed with respect to at least one or a combination of the following group of constituents: calcium, phosphorus, Lysomzyme, proteins, IgA, fat and/or the ones mentioned above in the summary of the invention. The analysis can be performed with known means and apparatus.

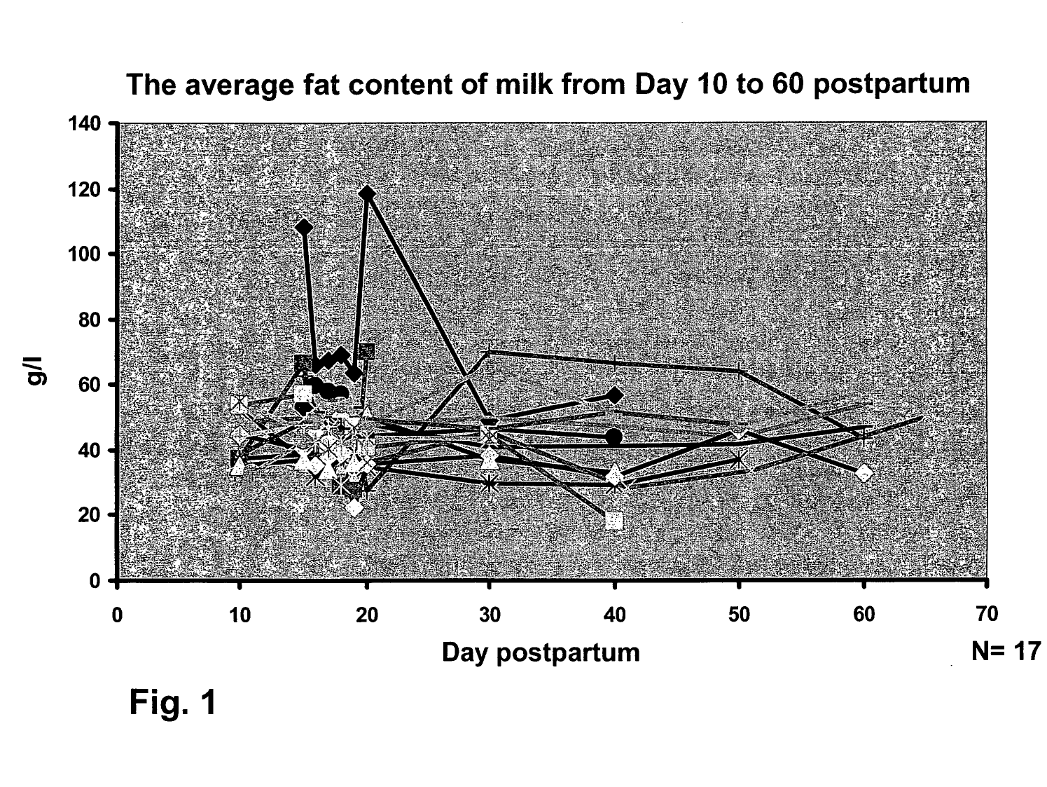

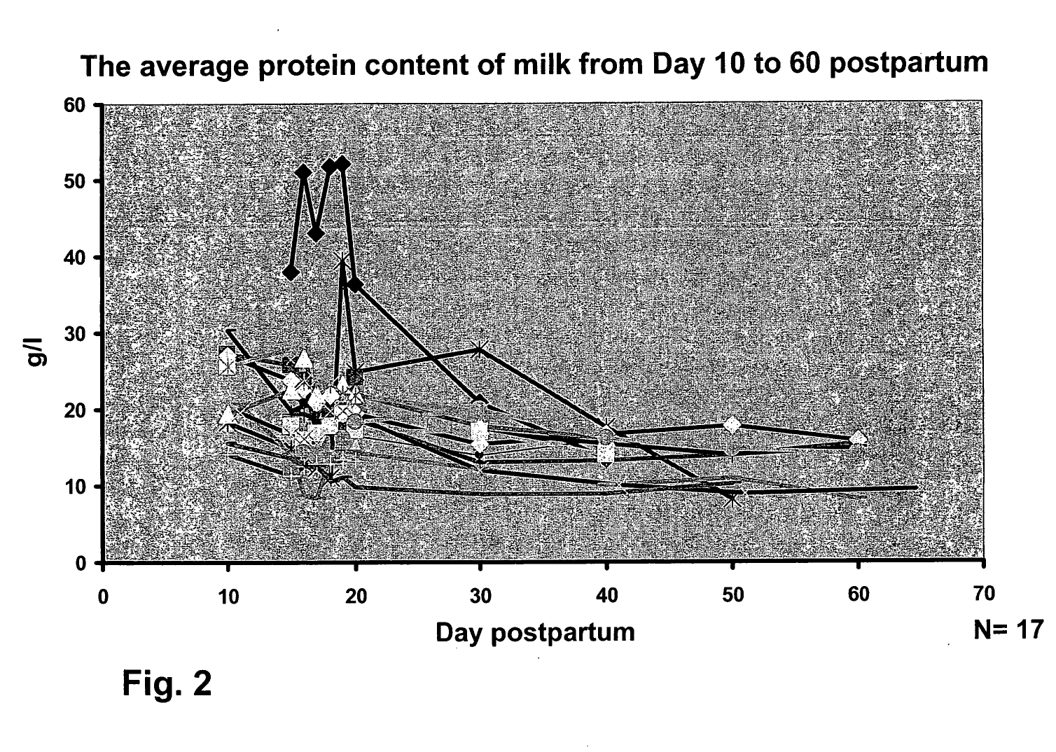

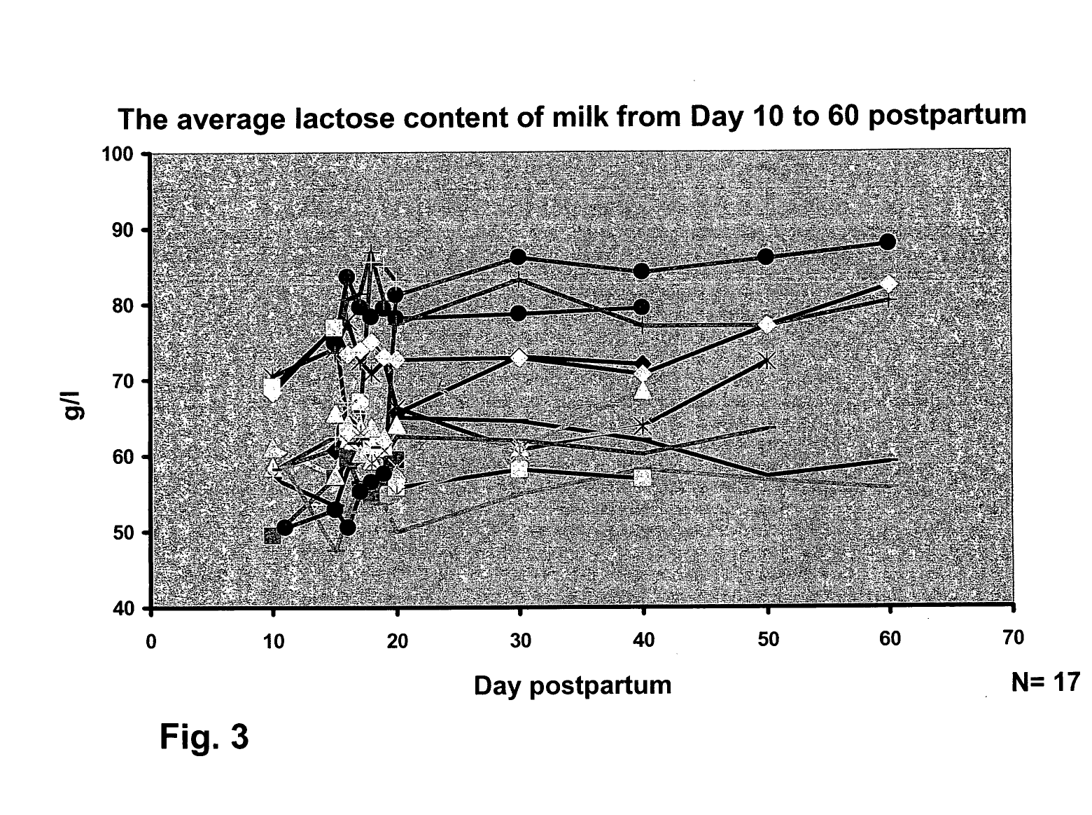

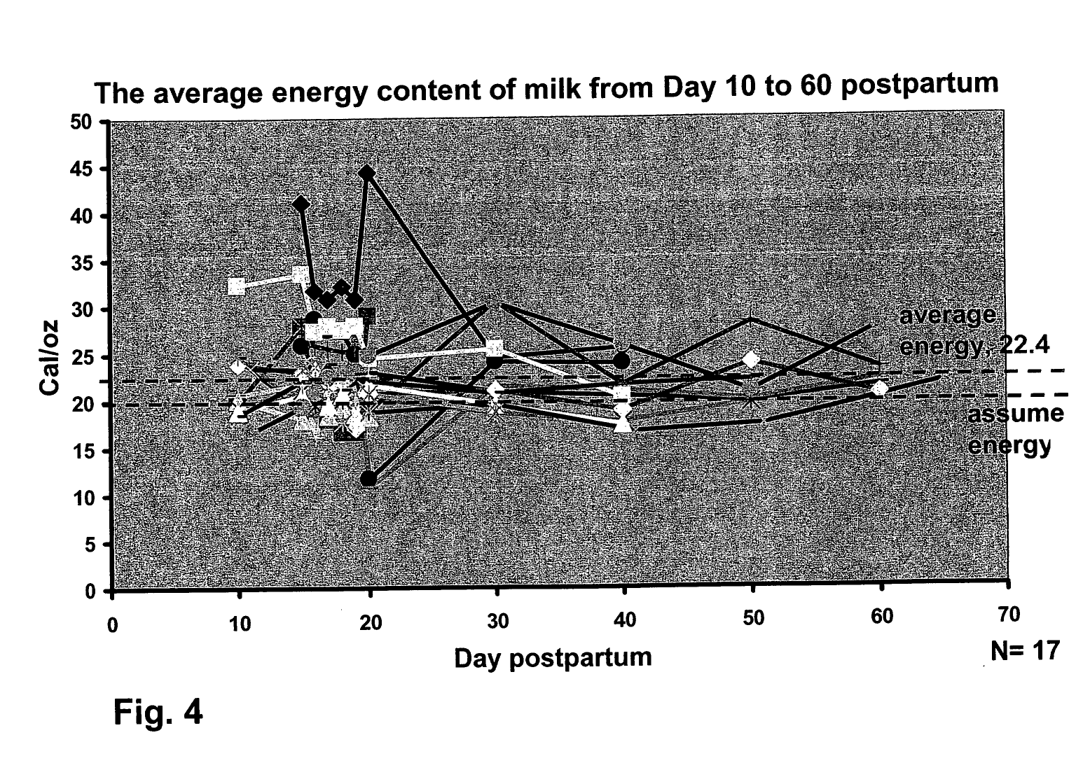

FIGS. 1 to 4 show the results of such analysis. FIG. 1 shows an average fat content of mother's milk from day 10 to 60 postpartum for different mothers. FIG. 2 shows an average protein content of mother's milk from day 10 to 60 postpartum for different mothers. FIG. 3 shows an average lactose content of mother's milk from day 10 to 60 postpartum for different mothers. FIG. 4 shows an average energy content of mother's milk from day 10 to 60 postpartum for different mothers.

The results of this analysis are used as a basis to decide, in which form the stored own mother's milk shall be fed to the infant. The form is thereby chosen from the group of unchanged own mother's milk, fortified own mother's milk, enriched mother's milk, deriched mother's milk. Enriched milk and deriched mother's milk can be for example milk, which lacks at least a part of some of its original constituents or which lacks at least a portion of such original constituents. If the infant has for example a lactose intolerance, the lactose in the milk can be reduced. This enriched or deriched milk can comprise supplements. However, it can also only consist of constituents which have already been present in the original collected milk.

The decision in which form the stored mother's milk shall be used is taken depending on at least some of said results of the analysis and said infant's condition. The data concerning the infants condition is chosen from at least one of the following parameters: infant's age, infant's weight, infant's health, infant's shortcomings, infant's deficiencies, time of day when the milk is fed to the infant.

If the mother's milk has on one day a volume of 300 ml and comprises 18.5 g lactose (74 cal), 2.4 g protein (9.6 cal) and 10.8 g fat (97 cal), then the total calories intake of the baby for this day is 180.6 cal or 18.6 cal/oz. The mother can then decide if this is sufficient for the baby or not. She can add fortifiers, enrich or derich the milk or feed it unchanged.

The own mother's milk can be tailored to fulfil the infants requirements. Different products can be obtained such as protein enriched milk, lactose free milk, sIgA rich milk.

For a preterm baby the recommended protein level is for example 2.5 g protein/kg/day, the recommended volume is <180 ml/kg/day and the recommended calories are 24 cal/oz. This means for a preterm baby of 1.730 kg a protein level of 4.3 g/day and a volume of 294 (300) ml per day.

Preferably, the mother's milk is analysed each time before it is fed to the infant. However, it is also possible to analyse the milk only periodically or at a special collecting time of day. For example, when the infant is always not sleeping well during the night, the mother's milk which is supposed to be usually last fed can be analysed. It is also possible, to analyse the milk only during special condition times of the infant, for example when the infant is ill or when he receives a new tooth. Special analysing programs and agendas depending on the mother and infant's conditions can be established for individual mothers and infants in order to meet their needs.

In a preferred embodiment of this invention, the results of the analysis are compared with recommended compositions of human milk which consider nutritional needs of infants with respect to at least some of the above mentioned infants conditions. These recommended compositions can be listed in at least one data table. This table can be printed on paper or on another appropriate medium. It can however also be stored in electronic form, for example on a disk which can be read by a data processing system. Preferably, there exist different tables comprising different recommended compositions for different infants conditions, at least a table for a healthy infants and a table for infants with short-comings or for preterm infants.

As a result of this comparison, changes are suggested which should be made to the collected mother's milk before it is fed to the infant. In a preferred embodiment of this invention, there exists at least one data table comprising possible compositions of human milk, which can occur when the mother's milk is collected. Additionally, the table preferably comprises data concerning changes to the collected own mother's milk to be made thereof to achieve these recommended compositions. For example, when the sampled milk is short of calcium, the changes suggested are the amount of calcium which should be added to the stored collected milk.

Instead of using tables or in addition thereto the inventive method comprises in one embodiment the step of running an optimisation computer program for finding an optimised composition based on the analysed sample, wherein the optimisation computer program uses as input data at least nutritional needs of infants in different conditions. The optimisation program can however take into respect other parameters, such as the time of day the milk shall be fed and/or the additional food the infant is obtaining during the day, the mother's health and the nutrient status.

The above mentioned tables are preferably all electronically stored for use in a computer system comprising data processing means, so that they can be processed automatically and so that a recommendation in which form the milk shall be used can be given automatically. The recommendation is preferably an output data naming at least one of the following: the supplement or supplements to be added to the milk, the way how the milk should be processed to be enriched or deriched, the information that the milk can be used as such.

In a preferred embodiment, an apparatus or system is provided which in addition or separately can perform the analysis of the sample automatically. If the analysing apparatus is not an integral part of the system processing the results, the apparatus preferably comprises means to forward the results to the system and the system comprises means to read the forwarded data. These means are known in the state of the art, they can comprise data wires or they can also be wireless, they can for example comprise infrared interfaces. However, it is also possible to enter the results manually using as input means for example a knob or a keypad or key board. The inventive system comprises means for comparing the evaluated recommended composition with these results of nutritional analysis and also means for evaluating changes to be made to the collected human milk in order to obtain a recommended composition. These means can be known data processing means used in common data processing devices and do not have to be described in detail. The system comprises output means for showing the suggested changes so that the mother or a support person knows what has to be done with the collected milk before it is fed. These output means can be a printout or a display or screen.

The system preferably also comprises a storage for storing the obtained results and/or suggested changes for comparison with said evaluated recommended composition. In a preferred embodiment, the system also considers former results and/or former suggested changes before suggesting a new change to a newly collected and analysed milk sample. This helps to compensate measuring and analysing errors. It also ensures, that the infant is not fed milk which varies too much during the day, if this is not a preferred task of the special infants nutrition.

### EXAMPLE 1

A liquid sample of human milk sample (100 ml) was taken. The human milk was taken from a mother of a preterm baby at >10 days and <90 days, respectively, after birth. The milk sample was taken from milk that had been expressed from the breast over the course of a day. The principal composition of this milk can be summarised as follows: 3.8% fat, 0.8% protein and 5.2% carbohydrate. The actual energy content of the milk sample was determined. The non-aqueous (cream) fraction of the milk was separated from the aqueous fraction by centrifugation at 10'000 rpm and a temperature of 4° C. and the top layer (the cream) was carefully removed and a known volume was added to 140 ml of the mother's own milk to increase the energy content of her milk to the recommended level for a preterm baby of the particular weight and age.

The preterm infant's growth and development could be observed to be similar to the one occurring in utero.

In addition, there may be a pasteurization and a standard hospital grade of bacteria assessment.

### EXAMPLE 2

A liquid sample of human milk sample (150 ml) was taken. The human milk was taken from a mother of a special need baby (preterm or sick term baby) at >10 days and <90 days, respectively, after birth. The milk sample was taken from milk that had been expressed from the breast over the course of a day. The principal composition of this milk can be summarised as follows: 3.8% fat, 0.8% protein and 5.2% carbohydrate. The concentration of protein in the milk sample was determined. The non-aqueous (cream) fraction of the milk was separated from the aqueous fraction by centrifugation at 10'000 rpm and 4° C. and the top layer (the cream) was carefully removed. The aqueous layer was then concentrated by passing it through a filter that was impervious to milk proteins. The filter used was a 30 Kd Omega Ultrafiltration Tangential flow filtration membrane from Pall. The filtration was performed at a low temperature, i.e. 19° C. The aqueous fraction is concentrated by performing a couple of 9 hours run tests on the final set up. A mean level of protein content is obtained which is concentrated in each hour and the final product is analysed in order to obtain a true protein content. Once the aqueous fraction had been concentrated 5 fold a known volume of the concentrated milk protein was added to 130 ml of mother's own milk to increase the protein content of the milk to the recommended level for a preterm baby or sick term baby of the particular weight and age.

The preterm infant's growth and development could be observed to be similar to the one occurring in utero and the term infant's growth and development could be observed to be similar to that of a term baby of a similar age.

In addition, there may be a pasteurization and a standard hospital grade of bacteria assessment.

### EXAMPLE 3

A liquid sample of human milk sample (500 ml) was taken. The human milk was taken from a human milk donor at <90 days after birth. The milk sample was taken from milk that had been expressed from the breast over the course of a few days and stored frozen. The principal composition of this milk can be summarised as follows: 3.8% fat, 0.8% protein and 5.2% carbohydrate. The concentration of protein in the milk sample was determined. The non-aqueous (cream) fraction of the milk was separated from the aqueous fraction by centrifugation at 10'000 rpm and 4° C. and the top layer (the cream) was carefully removed. The aqueous layer was then concentrated by passing it through a filter that was impervious to milk proteins. Preferably the same type of filter and temperature were used as in example 2. Once the aqueous fraction had been concentrated 5 fold in the same way as mentioned in example 2 the concentrated solution was centrifuged at high speed, i.e. 210'000 rpm and 4° C., to precipitate the casein fraction. The casein fraction, in addition to protein, contains a large proportion of the calcium and phosphorus that is in breast milk. This fraction was pasteurised at 60° C. for 35 minutes (hold method) and stored frozen either as a liquid or dried powder until required. Measured amounts of this enriched protein/calcium/phosphorus fraction was used to fortify mother's own milk for either preterm or sick term infants to restore their calcium and phosphorus balance. The preterm infant's growth and development could be observed to be similar to the one occurring in utero and the term infant's growth and development could be observed to be similar to that of a term baby of a similar age.

### EXAMPLE 4

A liquid sample of human milk sample (1000 ml) was taken. The human milk was taken from a mother of a preterm baby at >10 days and <90 days, respectively, after birth. The milk sample was taken from milk that had been expressed from the breast over the course of a few days and stored frozen. The principal composition of this milk can be summarised as follows: 3.8% fat, 0.8% protein and 5.2% carbohydrate. The concentration of protein in the milk sample was determined. The non-aqueous (cream) fraction of the milk was separated from the aqueous fraction by centrifugation at 10'000 rpm and 4° C. and the top layer (the cream) was carefully removed. The aqueous layer was then concentrated by passing it through a filter that was impervious to milk proteins. The same type of filter and temperature were used as in example 2. The aqueous fraction was concentrated 5 fold in the same way as mentioned in example 2, and then passed through an affinity column of Pharmacia to separate the sIgA and lysozyme. The sIgA and lysozyme solutions were then pasteurised at 60° C. for 35 minutes (hold method) and stored frozen as a liquid until required. Measured amounts of either the concentrated sIgA or lysozyme solutions were used to fortify the breast milk of mothers who had low concentrations of either sIgA or lysozyme in their milk.

The number of episodes of wheezing and respiratory tract infections in the first 12 months of life in the supplemented infants could be observed and compared to exclusively breastfed infants whose mothers have above average concentrations of sIgA and lysozyme in their milk.

It is possible to add human milk during ultrafiltration or to use reverse osmosis for further concentrate protein. Reverse osmosis has the advantage that it is faster than ultrafiltration.

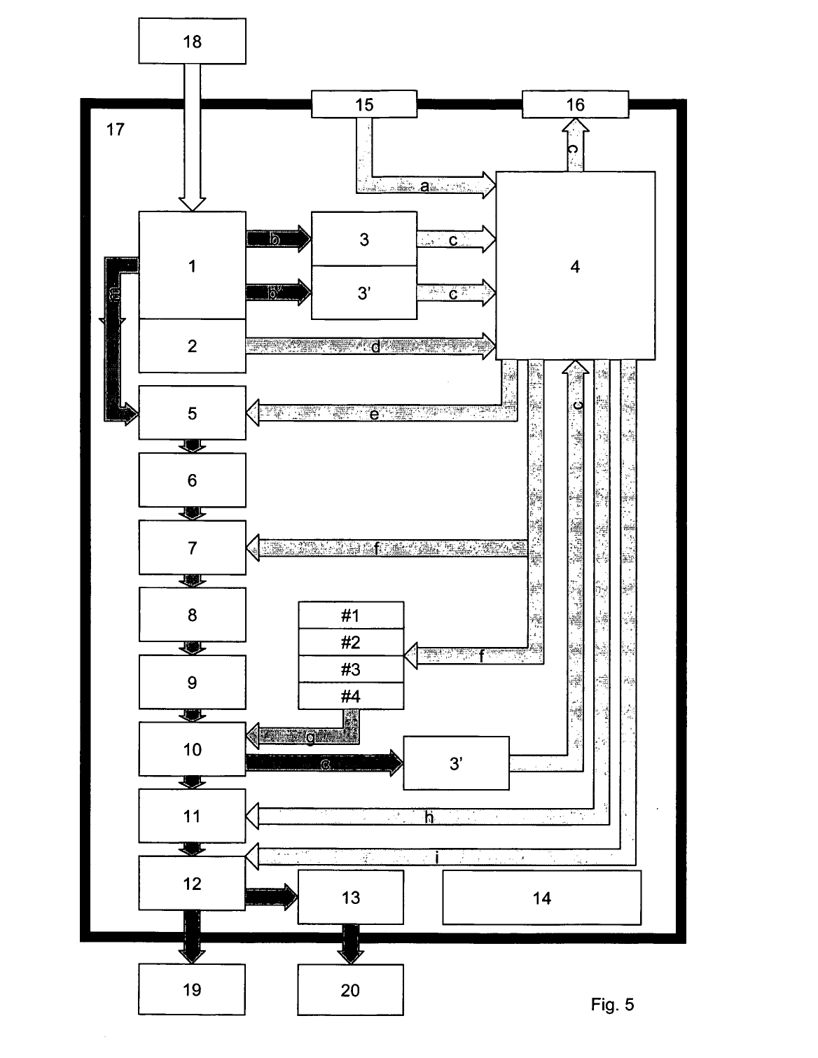

A particularly advantageous and fully integrated automated device for treatment of mother's milk is given in FIG. 5. This fortification or analysis device 17, which is provided in a single housing with corresponding input and output possibilities, comprises a data processing unit 4, including a keyboard for inputting data, as well as a memory slot for reading memory cards and a USB port for feeding in and out data.

Furthermore, the data processing unit 4 comprises a display, which may either be a full LCD screen, which may also be used as input device (touch screen), as well as either a connection for connecting to a printer or an integrated printing device.

Furthermore the data processing unit comprises a permanent memory in which general data are stored for calculating fortification schemes. These general data are basically given in the form of lookup tables, in which ideal milk compositions are given as a function of the condition of the baby and the mother. So for example for the different stages and health conditions of preterm babies, individual ideal compositions are stored in this database.

Differences from these ideal compositions as obtained by means of the analysis are interpreted and corresponding fortification and/or deriching schemes are calculated based on these differences.

The data processing unit therefore on the one hand serves to process the data obtained from the analysis units, to handle the data obtained about the mother and the child, and to calculate the necessary modifications scheme for the mother's milk. It furthermore is there to control the individual units or steps of the full process.

In addition the fortification device 17 is provided with the possibility of either introducing mother's milk (for example by means of tubing) or for directly introducing a container comprising mother's milk, which latter is designated with the reference numeral 1 in FIG. 5.

As already pointed out, all the subsequent steps after having introduced mothers milk into the device at 17, are carried out automatically and based on general input about ideal milk compositions and are based on individual information about the child and the mother.

The milk container 1 is provided with tubing or connections which allow to automatically take samples b for further analysis. These samples b are analysed in analysis units 3, 3′.

These analysis units are in this specific case given as one the one hand a spectrometer 3 which optically analyses the milk.

On the other hand there is provided an analyser 3′ for microbial analysis. The results c of these analysers 3, 3′ are automatically transferred to the data processing unit 4.

In addition to taking the samples the milk sample is weighted by means of a scale 2.

Since in particular the microbial analysis in many cases is time consuming, after the taking of the samples for analysis the milk is frozen for safe storage. This is schematically indicated by the reference numeral 5. However, freezing takes place using the same container as discussed above. So the surrounding of the container is provided with corresponding cooling circuits.

Depending on the result of the analysis and the subsequent interpretation of the data in the data processing unit, clearance e is given for fortification/deriching or not, the latter case occurring for example if the microbial analysis indicates heavy mastitis or the like.

If clearance e is given, and the analysis indicates that indeed a modification of the mother's milk is appropriate, subsequently the milk is thawed, which is schematically indicated with the reference numeral 6 in FIG. 5. Thawing may either take place applying heat (for example using the same circuit as used above for cooling), it may however also supported by mechanical treatment like crushing in order to handle the milk temperature wise as softly as possible. In this specific case, crushing is provided by means of a crushing arm introduced into the opening of the milk container.

Once the milk is at a temperature of around 0 and 10° C. and is in liquid state, and if the analysis comes to the conclusion that deriching of the milk is appropriate, the next step will be a deriching step schematically indicated with the reference numeral 7 in FIG. 5. Such a step may include centrifugation, filtering and the like, as is well-known in the field of treatment of mothers milk. Subsequently mixing and homogenisation takes place as the schematically indicated with the reference numeral 8.

After this treatment, or if this deriching step is not necessary directly after the above step 6, the milk sample is automatically pasteurised. To this end, it is heated up to the appropriate temperature for an appropriate amount of time. This step is schematically indicated with the reference numeral 9. After this step, and if the result of the data-processing indicates that fortification is necessary, different kinds of fortifiers indicated with #1, #2, #3 and #4 (see above) are introduced into the device. The fortifiers may be in solid state (powders) or they may be in liquid state. At the same time or just before introducing the fortifiers, an additional sample c is taken for further microbial analysis, to make sure that pasteurization has been carried out properly.

After this fortification, possibly after another mixing step, the milk is frozen (see reference numeral 11) if it is intended to keep it and store it for a certain time, and, if need be, the container is labelled in unit 12.

If the milk is going to be used directly, it will be directly output as indicated with reference numeral 20. If it is to be stored for a certain time in the device, it can also be frozen and kept in the device, and thawed automatically only shortly before use.

Generally, it is made sure in the device that the temperature in the milk never exceeds a temperature of 10° C. apart from the steps necessitating this like for example the pasteurising or possibly also the heating for unfreezing.

A particularly simple construction of such a device basically looks like a standard sample treatment apparatus with a possibility for introducing a container comprising mother's milk (using standard click on interfaces or the like), and a computer, which may either be integrated in the same housing or it may also be provided as a separate computer. In the latter case, the actual control in the analysis and treatment unit may be provided by a small control and data processing unit.

The advantage of such a device is certainly that the whole treatment of mothers milk can be carried out in an automated manner, making sure that best practices are applied, that sterile conditions are maintained, making sure that modifications of the milk are carried out in a standardised manner, and making sure that quick analyses and modifications are possible. The very specific order of steps as proposed in the above device makes sure that at any moment milk is handled safely preventing contaminations and microbial problems, that at the end milk is available which is properly fortified in a homogeneous state, and it makes sure that handling and modification can be carried out as quickly and efficiently as possible.

In particular for hospital use such a device is provided with a memory slot into which a data carrier of the corresponding mother can be introduced for a making sure that the milk is treated exactly the way as adapted to the mother's and the child's needs.

### LIST OF REFERENCE NUMERALS

1 milk collection, milk container

2 scale

3 analysis, in particular sensors, spectrometer etc

3′ analysis, in particular microbial analysis, in line or external

4 data-processing unit, including memory and lookup table

5 unit for storage and freezing

6 unit for thawing, in particular crusher, heating means

7 deriching unit, in particular centrifuge, filters

8 mixer/homogenizer

9 unit for pasteurisation

10 unit for storage at below 10° C.

11 unit for storage and freezing

12 unit for a labelling

13 unit for thawing, in particular crusher, heating means

14 tempering means to maintain temperature between 0 and 10° C.

15 keyboard, input device such as memory slot, disk drive

16 display and/or printer

17 fortification device

18 Mother's milk (input)

19 frozen milk for storage

20 milk ready to feed

a info about child/mother requirement to milk

b sample for analysis

b′ sample for microbial analysis

c results (e.g. data like spectra)

d weight

e clearance for fortification

f info about difference to ideal milk

g fortifiers

h clearance for feeding

i information (LOT number, date, composition etc)

1. A method for analysing and treating human milk to be fed to an infant comprising the steps of
collecting own mother's milk, taking a sample of the collected own mother's milk,
conducting nutritional analysis on said sample, using the collected own mother's milk as nutrition for the infant and
using the collected own mother's milk in the form of at least one of the group of: unchanged own mother's milk, fortified own mother's milk, enriched mother's milk, deriched mother's milk, wherein said form is chosen depending on at least some of said results of the analysis and said infant's condition, the infants condition being chosen from at least one of the following parameters: infant's age, infant's weight, infant's health, infant's shortcomings, infant's deficiencies, time of day when the milk is fed to the infant.

2. The method according to claim 1, comprising analysing and treating the own mother's milk periodically before feeding it to the infant.

3. The method according to claim 1, comprising analysing and treating the own mother's milk each time before feeding it to the infant.

4. The method according to claim 1, further comprising the steps of providing at least one data table, the at least one data table listing recommended compositions of human milk which consider nutritional needs of infants with respect to infants condition as well as listing possible compositions of human milk and changes to the collected own mother's milk to be made thereof to achieve these recommended compositions.

5. The method according to claim 4, further comprising the step of providing in this at least one data table different data of recommended compositions of human milk for nutrition of healthy infants and infants with diseases or shortcomings.

6. The method according to claim 1, further comprising providing a data processing apparatus for automatically processing the results of the analysis and automatically giving as output data a recommendation based on said results how to use the collected own mother's milk for nutrition of the infant.

7. The method according to claim 1, further comprising performing the analysis of the own mother's milk automatically.

8. The method according to claim 6 and 7, further comprising analysing the sample and processing the results in the same apparatus.

9. The method according to claim 1, further comprising analysing the sample with respect to at least one or a combination of the following group of constituents: calcium, phosphorus, lysomzyme, proteins, IgA, fat, protein and lactose, sIgA, lactoferrin, lysozyme, alpha-lactalbumin, serum albumin, beta casein, kappa casein, c3 and c4 complement, zinc, sodium, chloride, fatty acid composition, oligosaccharide

10. The method according to one of claims 1 to 9, further comprising running an optimisation program for finding an optimised composition based on the analysed sample, wherein the optimisation program uses as input data at least nutritional needs of infants in different conditions.

11. A method according to any of the preceding claims, wherein
mother's milk is collected after input, weighted and at least one sample taken for analysis, optionally including spectroscopic and/or microbial analysis,
the remaining mother's milk is stored and frozen,
the samples taken are analysed preferably by means of sensors (3, 3′) including spectroscopic sensors, microbial assays and the like,
the data of the analysis are input into a data processing unit (4) and therein compared with reference data available from a database, including general data as well as individual data about the baby to be fed, and determination of a mother's milk modification scheme,
if clearance is given based on the evaluation of the data of the analysis, the remaining mother's milk is thawed by heating and/or crushing,
based on the modification scheme, the remaining mother's milk is then
deriched by centrifugation and/or filtration and the like
mixed and/or, homogenized by sonication, pressure, jet injection and the like
and/or pasteurised
and/or fortified
and a sample taken for an additional, preferentially microbial analysis the modified mother's milk is subsequently frozen and stored, if need be labelled, and subsequently either kept in frozen state or thawed by means of heating and/or crushing and output for use for feeding,
all of the above steps being carried out automatically, and all of the steps being tempered to be at a temperature of not more than 10° C.

12. A system (17) for processing results of nutritional analysis of a sample of collected human milk comprising
a data processing device,
stored data related to recommended compositions of human milk which consider nutritional needs of infants in different conditions, the infants condition being 2 5 chosen from at least one of the following parameters: infant's age, infant's weight, infant's health, infant's shortcomings, infant's deficiencies, time of day when the milk is fed to the infant,
input means for entering data related to an infant to be fed with this collected human milk and
means for evaluating a recommended composition for said infant out of the group of said stored data related to recommended compositions,
means for comparing said evaluated recommended composition with said results of nutritional analysis,
means for evaluating changes to be made to the collected human milk in order to obtain a recommended composition and
output means for showing said changes to be made.

13. The system according to claim 12 wherein the system comprises input means for entering at least one of said results of the nutritional analysis of the sample.

14. The system according to claim 12 wherein the system comprises an apparatus for analysing the sample of human milk and wherein the results are stored in the apparatus for comparison with said evaluated recommended composition.

15. The system according to claim 12 wherein the systems comprising storing means for storing a history of the results and evaluated changes.

16. The system according to claim 13 wherein the system comprises stored data tables possible compositions of human milk.

17. The system according to claim 16 wherein the system comprises stored data related to possible changes to be made to the possible composition of human milk in order to achieve recommended compositions.

18. The system according to one of claims 12 to 17 wherein the system comprises stored data of recommended compositions of human milk for nutrition being specifically adopted for healthy infants and for infants with diseases or shortcomings.

19. Device (17) according to any of claims 12-18, comprising a milk container (1) with a scale or other means (2) for weighting milk contained in said milk container (1),
analysing means (3, 3′) for taking samples from said milk container (1) and for analysing the samples;
a data processing unit (4) for interpreting the data obtained from the analysing means (3, 3′) for the determination of a modification scheme for the mother's milk, and for controlling further handling and fortification of the remaining mother's milk;
a first unit (5) for storage and freezing during analysis of the remaining mother's milk;
a first unit (6) for thawing the remaining mother's milk, preferably including heating and/or crushing means;
a unit (7) for deriching the remaining mother's milk, preferably including centrifugation and/or filtering means;
a unit (8) for homogenisation of the deriched mother's milk, preferably including sonication, pressure and/or jet injection means;
a unit (9) for pasteurization;
a unit (10) for fortification including input means for at least one fortifier and including means for taking at least one sample for additional microbial analysis;
a second unit (11) for storage and freezing;
optionally a unit (12) for labelling of samples;
a second unit (13) for thawing of the modified mother's milk, preferably including heating and/or crushing means;
an output device for the modified mother's milk.

20. Device according to claim 19, wherein the analysing means include microbial analysis and/or a spectrometer.

21. Device according to claim 19 or 20, wherein tempering means (14) are provided such that in the milk container and in each unit a temperature of not more than 10° C. is safeguarded.

22. Device according to any of claims 19-21, wherein transport of the milk between the units (5-13) is provided by means of tubing, wherein preferentially means are provided to wash said tubing between individual transfer steps of mother's milk.

23. Device according to any of claims 19-22, wherein transport of the milk between the units (5-13) is provided by means of a standardised container, which is subject to different connections and/or inputs.

24. Device according to claims 19-23, wherein data input means (15) are provided, which are preferentially given by a keyboard and/or by an interface such as a memory slot, and wherein data output means (16) are provided, which are preferentially given by a printer and/or a display.
